SEQUENCE LISTING (7 sequences)

SEQ ID NO: 1 - DNA, 39 bp, Artificial Sequence
  Synthesized in laboratory
gcggcgacctgaagttgcttggctggtgtgaagaaggag

SEQ ID NO: 2 - DNA, 20 bp, Artificial Sequence
  Synthesized in laboratory
ggggaatacactacgtgact

SEQ ID NO: 3 - DNA, 19 bp, Artificial Sequence
  Synthesized in laboratory
ccgggtagtttccaactcc

SEQ ID NO: 4 - DNA, 20 bp, Artificial Sequence
  Synthesized in laboratory
ttccccggggatccctcttc

SEQ ID NO: 5 - DNA, 22 bp, Artificial Sequence
  Synthesized in laboratory
cggcttcttcttgttcgcgtcc

SEQ ID NO: 6 - DNA, 38 bp, Artificial Sequence
  Synthesized in laboratory
tctccgcctcgctcgagtagctgtatggtggcaagggc

SEQ ID NO: 7 - DNA, 10 bp, Artificial Sequence
  Synthesized in laboratory
gaggcggaga

(19) United States
(12) Patent Application Publication
(10) Pub. No.: US 2025/0066865 A1
(43) Pub. Date: Feb. 27, 2025

(54) DETECTION OF PATHOGENS IN PLANTS

(71) Applicant: Purple City Labs, Inc., Oakland, CA (US)

(72) Inventors: ALI BEKTAS TONAK, OAKLAND, CA (US); KJEL MASTELLOS-JOHNSON, Oakland, CA (US)

(21) Appl. No.: 18/546,828

(22) PCT Filed: Feb. 18, 2022
(86) PCT No.: PCT/US2022/070732

§ 371 Date: Aug. 17, 2023

Related U.S. Application Data
(60) Provisional application No. 63/151,462, filed on Feb. 19, 2021.

(51) Int. Cl. C12Q 1/70; C12Q 1/6806; C12Q 1/6844; G01N 1/28; G01N 21/64

(52) U.S. Cl. CPC C12Q 1/701; C12Q 1/6806; C12Q 1/6844; G01N 1/286; G01N 21/6428; G01N 2001/2866; G01N 2021/6439

(57) ABSTRACT

The invention relates to reagents, compositions, methods, and kits for detecting a hop latent viroid in a plant.

### Claim OF PRIORITY UNDER 35 U.S.C. § 119

The present application is a U.S. National Stage entry under U.S.C. 371 of International Application No. PCT/US2022/070732, filed on Feb. 18, 2022, designating the United States of America and published in English on Aug. 25, 2022, which in turn claims benefit of Provisional Application No. 63/151,462, entitled “DETECTION OF PATHOGENS IN PLANTS” filed Feb. 19, 2021, the entire contents of each of the foregoing is hereby incorporated by reference herein.

### REFERENCE TO AN ELECTRONIC SEQUENCE LISTING

The contents of the electronic sequence listing (2024-03-07-Corrected Sequence Listing_ST25-PUCG-005US0.xml; Size: 1,692 bytes; and Date of Creation: Feb. 6, 2024) is herein incorporated by reference in its entirety.

### BACKGROUND

Field

The invention provided herein relates to reagents, compositions, methods, and kits for detecting hop latent viroid in a plant.

Background

Cannabis plants can be susceptible to infection by pathogens. Pathogens can include viruses, viroids, bacteria, fungi, nematodes, and/or any organisms that can cause disease in plants. Certain pathogens can reduce the quality and/or productivity of plants, and in certain instances, pathogens can cause plant death. Pathogens can be introduced and spread to host plants in a variety of ways. For example, bacterial and fungal spores can be transmitted by wind, rain, and/or soil. Certain pathogens can be spread through insects, transplants, infected seeds, irrigation water, contaminated equipment, and humans.

One pathogen capable of infecting Cannabis plants is the hop latent viroid (HLVd). Symptoms of a HLVd infection may include reduction or lack of oil, small heads, misshapen leaves, leaves that are yellowish in color, brittle stems, an outwardly horizontal plant structure, and reduced flower mass and trichomes, although some plants infected with HLVd or a HLVd variant may be asymptomatic. Other pathogens with similar deleterious effects include viruses such as Beet Curly Top Virus (BCTV) and Alfalfa Mosaic Virus (AMV). Given the potentially detrimental effects of HLVd infection and viruses such as BCTV and AMV in Cannabis plants, there is a need for accurate diagnostics of HLVd and/or other pathogenic infection; for an assessment of the relationship between HLVd or other pathogenic variants; and presentation of symptoms.

### SUMMARY

Some embodiments of the invention relate to a method of detecting hop latent viroid in a plant sample. The method can include: obtaining a plant sample; processing the plant sample in an extraction buffer; and/or amplifying a target nucleic acid sequence from processed plant sample using a loop-mediated isothermal amplification (LAMP) reaction. The LAMP reaction can be conducted with one or more primers selected from SEQ ID NOs 1-7. The reaction can produce a detectable signal, where the signal can indicate the presence of the target nucleic acid, and where the presence of the target nucleic acid can be indicative of hop latent viroid in the plant sample.

In some embodiments, the processing step can include pulverizing the plant sample in the extraction buffer to obtain a pulverized sample.

In some embodiments, the extraction buffer can include a visual aid, such as a dye. In some embodiments, the dye is phenol red.

In some embodiments, the amplifying step can include contacting the processed plant sample with detection reagents.

In some embodiments, the detection reagents can include freeze-dried reaction beads.

In some embodiments, the signal is a fluorescent signal.

In some embodiments, the plant is a Cannabis plant.

In some embodiments, the plant sample can be stem, leaf, sap, root, flower,, and/or the like.

Some embodiments of the invention relate to a kit for a method disclosed herein. The kit can include at least one reagent.

### BRIEF DESCRIPTION OF THE DRAWINGS

FIG. 1 depicts results from samples tested in methods disclosed herein.

### DETAILED DESCRIPTION

Described herein are methods, reagents, and kits for detecting a pathogen in a plant.

### Methods

Methods of the invention can relate to detecting a pathogen in a plant by detection of an analyte. An exemplary analyte can include one or more of: antigens, receptors, proteins, peptides, nucleic acids, sugars, lipid, carbohydrates, glycans, glycoproteins, oligonucleotides, cells, viruses, viroids, and any combinations thereof. In some embodiments, the nucleic acids can include, e.g., cellular DNA or RNA, messenger RNA, microRNA, ribosomal RNA, and any combinations thereof.

The method can include obtaining a plant sample; processing the plant sample in an extraction buffer; and amplifying a target nucleic acid sequence from the processed plant sample using an isothermal reaction, where the reaction can produce a detectable signal and where the signal can indicate the presence of the target nucleic acid. The presence of the target nucleic acid can be indicative of a pathogen in the plant sample.

In some embodiments, following sampling, stem tissue of the plant can be treated with rooting hormone and placed conditions for rooting. In this way, testing can integrate into the production line rather than be its own independent procedure.

In the methods and compositions provided herein, in some embodiments, the polynucleotide primer pairs are designed to specifically hybridize to and amplify a subsequence of the nucleic acid of the pathogen that is non-identical to any subsequence of the nucleic acid of the plant genome, thereby permitting specific detection of the plant pathogen and avoiding non-specific detection of sequences of the plant nucleic acid.

### Plant Sample

The plant sample used in the methods disclosed herein can be from any plant part or component, including, but not limited to meristem, bud, stem, leaf, sap, roots, pollen, seed or flower.

The plant can be a Cannabis plant, other members of the cannabaceae such as Humulus lupus, and or the like.

### Pathogen

The term “virus,” as used herein, refers to an infective particle comprising nucleic acid and protein, wherein the particle multiplies by infecting a host organism, such as a plant or animal, that is different from the virus. A “viroid,” as used herein, refers to an infective particle consisting of RNA without an accompanying protein coat/component, typically smaller than a virus. Viroids, like viruses, can multiply by infecting a host organism that is different from the viroid. Both “virus” and “viroid,” as used herein, are terms known to and understood by those of skill in the art.

The pathogen being detected can be HLVd.

### Processing/Extraction

In some embodiments, the method can include processing the plant sample with an extraction buffer. In some embodiments, the method can include mixing the sample and extraction buffer by any means known in the art such as agitating manually or mechanically with a tissue homogenizer. In some embodiments, beads are used to aid with homogenization. The beads can be zirconium, ceramic, steel, glass, and/or the like. The beads can be of appropriate diameter such as 1-5 mm, for example about 1, 2, 3, 4, or 5 mm. In other embodiments, a pestle can be used to crush the tissue. The mixing can occur for a time sufficient to substantially homogenize the mixture. The incubation time and temperature conditions sufficient to produce a homogenized sample will be readily determined be one of ordinary skill in the art and will depend in part on the requirements of the particular reagents chosen. For example, the sample can be mixed for 1, 2, 5, 10, min, or more at temperatures between about 25° C. and about 40° C. or more, or about between 30-37° C.

As used herein “homogenized sample” and “homogenate” refer to a suspension of cell fragments and cell constituents that is substantially uniform or similar.

As used herein, the term “substantially” modifies a particular value, by referring to a range equal to the particular value, plus or minus ten percent (+/−10%).

The extraction buffer can be a modified Tris-HCL buffer consisting of 100 mM Tris-HCL, pH 8.0m, 50 mM EDTA, and 100 mM NaCl. Alternative extraction buffers known to those of skill in the art, such as but not limited to 0.1 M NaOH, QuickExtract™ (Lucigen), or a TCEP/EDTA-modified Heating Unextracted Diagnostic Samples to Obliterate Nucleases (HUDSON) buffer can also be used. In some embodiments, detergents such as SDS or CTAB can be used.

In some embodiments, depending on the buffer utilized, the method can include a further dilution step with RNase-free molecular biology grade water in order to reduce the concentration of Mg chelating agents such as EDTA.

In some embodiments, the extraction buffer can include a dye such as phenol red or the like. The dye can act as a visual cue throughout the method to ensure that pipetting and diluting steps are performed accurately. This dye can also act as a secondary colorimetric indicator for the LAMP reaction. Further details can be found in Tanner NA, Zhang Y, Evans TC Jr. Visual detection of isothermal nucleic acid amplification using pH-sensitive dyes. Biotechniques. 2015 Feb. 1;58 (2): 59-68, which is fully incorporated by reference herein.

### Amplifying

In some embodiments, the method can include amplifying a target nucleic acid sequence from a processed plant sample using an isothermal reaction. Amplification techniques can include rolling circle amplification (RCA), recombinase polymerase amplification (RPA), strand displacement amplification (SDA), and loop-mediated isothermal amplification (LAMP). Other examples of amplification techniques can include nucleic acid sequence-based amplification (NASBA), transcription mediated amplification (TMA), helicase dependent amplification (HDA), and the like. Regarding LAMP in particular, it is a powerful isothermal nucleic acid amplification technique that can generate approximately 10⁹ copies from less than 10 copies of template DNA within an hour or two. In some embodiments, the isothermal reaction can be LAMP.

In some embodiments, the amplifying step can include contacting the processed plant sample with detection reagents. Detection reagents can include primers, reagents, reaction beads, fluorescent intercalating dyes, pH indicators, conjugated antibodies and appropriate buffers.

The detection reagents can be in the form of reaction beads. The reaction beads can include primers, a DNA polymerase (e.g., with 5′ to 3′ strand displacement activity such as Bst LF, or a modified version), a reverse transcriptase, dNTPs, a suitable buffer for enzymatic activity, an intercalating dye, and/or the like. The reaction beads can be freeze-dried.

In some embodiments, the method can be conducted with 2, 3, 4, 5, 6, 7, 8 or more primers. Exemplary primers are provided in Table 1:

TABLE 1
Exemplary primers

| Primer Name | Primer Sequence |
|---|---|
| BIP | GCGGCGACCTGAAGTTGCTTGGCTG |
| | GTGTGAAGAAGGAG (SEQ ID No. 1) |
| F3 | GGGGAATACACTACGTGACT (SEQ ID No. 2) |
| B3 | CCGGGTAGTTTCCAACTCC (SEQ ID No. 3) |
| LF | TTCCCCGGGGATCCCTCTTC (SEQ ID No. 4) |
| LB | CGGCTTCTTCTTGTTCGCGTCC (SEQ ID No. 5) |
| FIP-FAM | /56-FAM/TCTCCGCCTCGCTCGA |
| | GTAGCTGTATGGTGGCAAGGGC (SEQ ID No. 6) |
| FIP-Q | GAGGCGGAGA (SEQ ID No. 7)/3IABkFQ/ |

“/56-FAM/” is fluorescein
“/3IABkFQ/” is Iowa Black Quencher
Other quenchers such as fluorescent TAMRA ™ or nonfluorescent Black Hole Quencher ™ (BHQ ™) can be used.

Incubation conditions are generally 65C for one hour. The level of sensitivity of the reaction can be increased 10-fold by a pre-incubation, for example but not limited to a pre-incubation of 12 minutes at 55C, which is the optimum temperature for the enzymatic activity of the preferred reverse transcriptase WarmStart RtX. When using other reverse transcriptases, as are known in the art, different pre-incubation conditions can be use as needed to optimize reverse transcriptase efficiency. Overall, the periods of time and temperature conditions disclosed here can vary. For example, incubation conditions can be at about 50C, 55C, 60C, 70C, or 75C for 45, 50, 55, 65, 70, 75 min or more. For example, pre-incubation can be for 5, 6, 7, 8, 9, 10, 11, 13, 14, 15, 16, or more minutes at 45C, 50C, 60C, 65C, or 70C.

### Detection

Reaction reagents can include various means for producing a detectable signal. In some embodiments, the detectable signal can include but need not be limited to an optical label such as a small-molecule dye, a fluorescent molecule or protein, a quantum dot, a colorimetric reagent, a chromogenic molecule or protein, a Raman label, or the like, and any combinations thereof. In some embodiments, the detectable label or optical label can be or include a fluorescent molecule or protein.

It is also possible to use other methods of reporting amplification in a non-sequence specific manner. These include, but are not limited to, pH-based reporting such as with phenol red, Mg2+ ion-based reporting such as with hydroxynaphthol blue (HNB), electrochemical methods, observation of turbidity resulting from phyrophosphate accumulation, or the use of DNA intercalating dyes such as EvaGreen® or SYBR® Green I. In these embodiments, the FIP-Q oligonucleotide is omitted and a non-modified version of FIP-FAM is used (e.g. TCTCCGCCTCGCTCGAGTAGCTGTATGGTGGCAAGGGC (SEQ ID NO. 6). FIP-FAM is a fluorescently modified oligonucleotide following the design parameters of QUASR from Sandia labs. If not incorporated it is quenched by a complementary, yet shorter oligo modified with a quencher. By omitting the fluorophore (FAM) an intercalating dye can be used to report amplification, in a non-sequence specific manner.

### Kits

The invention can also include kits to carry out the methods disclosed herein. The kit can include reaction reagents, collection tubes, an extraction buffer, DEPC treated water for dilution of the sample, glass or zirconium beads and/or plastic pestles for macerating the sample. Additional components can be fixed volume Pasteur pipettes for diluting the sample and/or moving it on to the reaction beads, an incubator with or without a built-in excitation light source, appropriate emission filters, and/or the like.

### EXAMPLES

### Example 1

Samples from a Cannabis plant infected with HLVd were compared with samples from an uninfected Cannabis plant. The samples were stem or petiole tissue of approximately 1 cm in length. Each sample was placed into a 1.5 ml microcentrifuge tube prefilled with 200 μl extraction buffer and 3 glass beads of 3 mm diameter. Following sampling, the stem tissue cut was treated with rooting hormone and placed in a rockwool cube for rooting.

The extraction buffer was modified Tris-HCL buffer consisting of 100 mM Tris-HCL, pH 8.0m, 50 mM EDTA, and 100 mM NaCl. The sample in extraction buffer with glass beads was pulverized in a bead beater and then briefly spun down to pellet cell debris. The supernatant was diluted at 1:50 ratio in order to minimize inhibitory metabolites and/or autofluorescent compounds.

2-5 μM of the diluted sample preparation was directly added to a RT-LAMP reaction mixture consisting of the following primers and concentrations: 1.6 μM FIP-F, 1.6 μM BIP, 0.2 μM F3, 0.2 μM B3, 0.4 μM LF, 0.4 μM LB, and 2.3 μM FIP-Q. The reaction mixture was made up in 10X isothermal amplification buffer II (NEB), 12 units of Bst 3.0 (NEB), 7.5 units of Warm Start RTx (NEB), 1.5 mM dNTPs and 4.5 mM total MgSO4.

The reaction uses the QUASR (for quenching of unincorporated amplification signal reporters) method of sequence-specific end point fluorescence of a visual signal. Further details can be found in Ball CS, Light YK, Koh CY, Wheeler SS, Coffey LL, Meagher RJ. Quenching of Unincorporated Amplification Signal Reporters in Reverse-Transcription Loop-Mediated Isothermal Amplification Enabling Bright, Single-Step, Closed-Tube, and Multiplexed Detection of RNA Viruses. Anal Chem. 2016 Apr. 5;88 (7): 3562-8. doi: 10.1021/acs.analchem.5b04054. Epub 2016 Mar. 24. PMID: 26980448, which is fully incorporated by reference herein. The primers used were as follows:

| | |
|---|---|
| BIP | GCGGCGACCTGAAGTTGCTTGGCTGGTGTGAAGAAGGAG |
| | (SEQ ID No. 1) |
| F3 | GGGGAATACACTACGTGACT (SEQ ID No. 2) |
| B3 | CCGGGTAGTTTCCAACTCC (SEQ ID No. 3) |
| LF | TTCCCCGGGGATCCCTCTTC (SEQ ID No. 4) |
| LB | CGGCTTCTTCTTGTTCGCGTCC (SEQ ID No. 5) |
| FIP-FAM | /56-FAM/TCTCCGCCTCGCTCGAGTAGCTGTATGGTGG |
| | CAAGGGC (SEQ ID No. 6) |
| FIP-Q | GAGGCGGAGA (SEQ ID No. 7)/3IABkFQ/ |

Reactions were incubated at 65° C. for 45-60 min. After allowing the reaction vessel to cool down, the incubation tubes were excited with a light source.

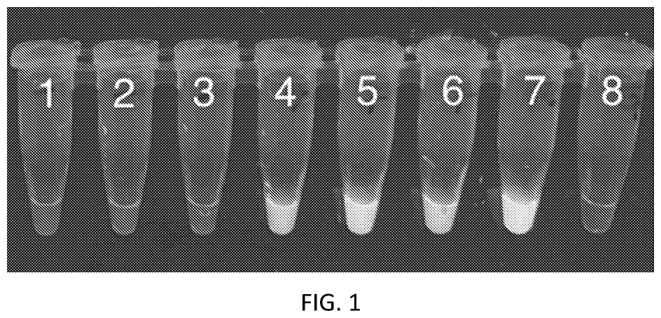

Results are shown in FIG. 1 and correspond with following from left to right.

1. Crude prep from uninfected plant 1

2. Crude prep from uninfected plant 2

3. Crude prep from uninfected plant 3

4. Crude prep from infected plant 1

5. Crude prep from infected plant 2

6. Crude prep from infected plant 3

7. Purified RNA from infected plant

8. Non-template water control

The green fluorescence present in samples tubes 4-7 indicates amplification and thus a positive for HLVd due to the incorporation of the fluorescently tagged FIP primer into LAMP amplicons.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques.

Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. 1. (Original) A method of detecting hop latent viroid in a plant sample, the method comprising
a. obtaining a plant sample;
b. processing the plant sample in an extraction buffer; and
c. amplifying a target nucleic acid sequence from processed plant sample using a loop-mediated isothermal amplification (LAMP) reaction, wherein the LAMP reaction is conducted with one or more primers selected from SEQ ID NOs 1-7;
wherein the reaction produces a detectable signal, wherein the signal indicates the presence of the target nucleic acid, and wherein the presence of the target nucleic acid is indicative of hop latent viroid in the plant sample.

2. The method of claim 1, wherein the processing step comprises pulverizing the plant sample in the extraction buffer to obtain a pulverized sample.

3. The method of claim 2, wherein the extraction buffer includes a visual aid, wherein the visual aid is a dye.

4. The method of claim 4, wherein the dye is phenol red.

5. The method of claim 1, wherein the amplifying step comprises contacting the processed plant sample with detection reagents.

6. The method of claim 5, wherein the detection reagents comprise freeze-dried reaction beads.

7. The method of claim 1, wherein the signal is a fluorescent signal.

8. The method of claim 1, wherein the plant is a Cannabis plant.

9. The method of claim 1, wherein the plant sample is selected from stem, leaf, sap, root, or flower.

10. A kit for the method of claim 1 comprising a reagent.
